(19) United States
(12) Patent Application Publication
(10) Pub. No.: US 2026/0348956 A1
(43) Pub. Date: Nov. 5, 2026

(54) BIOMARKER FOR PREDICTING SYSTEMIC LUPUS ERYTHEMATOSUS FLARE

(71) Applicants: AJOU UNIVERSITY INDUSTRY-ACADEMIC COOPERATION FOUNDATION, Suwon-si (KR); Industry-University Cooperation Foundation Hanyang University, Seoul (KR)

(72) Inventors: Kyu-Tae Kim, Suwon-si (KR); Sang-Cheol Bae, Seoul (KR); Hye-Soon Lee, Seoul (KR); Seung-Jun Paek, Suwon-si (KR)

(21) Appl. No.: 19/712,023

(22) Filed: Jun. 18, 2026

Related U.S. Application Data
(63) Continuation of application No. PCT/KR2024/018958, filed on Nov. 27, 2024.

(30) Foreign Application Priority Data
Dec. 28, 2023 (KR) 10-2023-0194854
Jul. 24, 2024 (KR) 10-2024-0098190

(51) Int. Cl. G01N 33/564; C12Q 1/6883

(52) U.S. Cl. CPC G01N 33/564; C12Q 1/6883; C12Q 2600/158; G01N 2800/52

(57) ABSTRACT

Provided is a biomarker for predicting systemic lupus erythematosus (SLE) flare. A biomarker composition includes, as an active ingredient, a protein expressed in CD8+ T cells, or a gene encoding the protein, wherein the protein or the gene may include one or more of GZMH, GZMB, GZMA, GNLY, and PRF1. Also provided are a composition and a kit including an agent capable of measuring an expression level of the protein or the gene, a method of predicting an SLE flare by isolating CD8+ T cells from a blood sample and comparing expression levels with those of a control sample, and a method of screening a therapeutic agent for SLE. When upregulation of the expression level is observed, a subject is determined to be likely to experience an SLE flare, enabling more accurate prediction than conventional biomarkers.

### FIELD

The present disclosure relates to a biomarker for predicting systemic lupus erythematosus (SLE) flare.

### DESCRIPTION OF THE RELATED ART

Systemic lupus erythematosus (SLE) is a representative systemic autoimmune disease in which various immune cells are involved in its pathogenesis. Clinical symptoms of SLE vary widely, ranging from mild skin and joint diseases to life-threatening severe organ complications. Patients with SLE exhibit individually variable and unpredictable disease courses, which suggests cellular and molecular heterogeneity reflecting patient-specific variations at genetic, epigenetic, transcriptomic, and proteomic levels.

The main therapeutic goals in managing SLE are to achieve remission or reduce disease activity, prevent organ damage, minimize drug toxicity, and improve quality of life. However, due to the complex pathophysiological characteristics of SLE, treatment is difficult, and therapeutic responses may vary considerably among patients. Therefore, a personalized approach based on patient stratification is essential for effective disease management.

Since multiple types of immune cells are involved in the pathogenesis of SLE, profiling peripheral blood mononuclear cells (PBMCs) serves as a powerful tool for understanding disease trajectories and improving patient outcomes. Indeed, gene expression profiling studies of bulk immune cells from SLE patients have revealed distinct phenotypic characteristics of patient subgroups, which may potentially improve patient stratification. However, an approach that collectively considers heterogeneous immune cells as an integrated population may not fully capture the complete immune landscape of SLE, and therefore may overlook subtle but important clinical implications of cell subpopulations related to treatment.

To overcome these limitations and provide a detailed view of individual cellular transcriptomes, single-cell RNA sequencing (scRNA-seq) has been applied to profile PBMCs from SLE patients. These studies have led to several important findings by dissecting the heterogeneity of functional behaviors of immune cells within and among SLE patients. For example, identification of more than 20 distinct cell subpopulations actively involved in disease pathogenesis, global expression of interferon gene signatures in specific cell types, and differences in specific cellular compositions compared with controls have been revealed.

However, many conventional scRNA-seq studies have merely provided snapshots through cross-sectional approaches, and the evolving characteristics of immune cells during disease progression remain largely unexplored. Clarifying cellular and molecular changes during various disease stages, particularly during flare and remission periods, is essential for fully understanding the underlying pathogenesis of SLE.

Accordingly, there is a need for a new method capable of tracking dynamic changes in immune cells according to the disease course of patients with systemic lupus erythematosus and clarifying cellular and molecular changes during flare and remission periods.

Korean Patent Registration No. 10-2475926, which is a background art of the present disclosure, relates to a biomarker composition for diagnosing systemic lupus erythematosus and a method for providing information necessary for diagnosing systemic lupus erythematosus using the same.

### DESCRIPTION

### Technical Problem

The present disclosure is directed to solving the problems of the prior art described above and provides a biomarker composition for predicting systemic lupus erythematosus (SLE) flare.

The present disclosure also provides a composition for predicting systemic lupus erythematosus flare.

The present disclosure also provides a kit for predicting systemic lupus erythematosus flare, comprising the composition for predicting systemic lupus erythematosus flare.

The present disclosure also provides a method for predicting systemic lupus erythematosus flare.

The present disclosure also provides a method for screening a therapeutic agent for systemic lupus erythematosus.

However, the technical problems to be achieved by the embodiments of the present disclosure are not limited to the technical problems described above, and there may be other technical problems.

### Means for Solving the Problem

As a technical means for achieving the aforementioned technical problems, a first aspect of the present disclosure provides a biomarker composition for predicting systemic lupus erythematosus (SLE) flare, comprising a protein expressed in CD8+ T cells or a gene encoding the same as an active ingredient.

According to one embodiment of the present disclosure, the protein expressed in the CD8+ T cells or the gene encoding the same may comprise one or more selected from the group consisting of GZMH, GZMB, GZMA, GNLY, and PRF1, but is not limited thereto.

According to one embodiment of the present disclosure, when upregulation of an expression level of one or more of GZMH, GZMB, GZMA, GNLY, and PRF1 is observed, a subject may be determined to be likely to experience a systemic lupus erythematosus flare, but is not limited thereto.

In addition, a second aspect of the present disclosure provides a composition for predicting systemic lupus erythematosus (SLE) flare, comprising an agent capable of measuring expression levels of a protein expressed in CD8+ T cells or a gene encoding the same as an active ingredient.

According to one embodiment of the present disclosure, the protein expressed in the CD8+ T cells or the gene encoding the same may comprise one or more selected from the group consisting of GZMH, GZMB, GZMA, GNLY, and PRF1, but is not limited thereto.

According to one embodiment of the present disclosure, when upregulation of an expression level of one or more of GZMH, GZMB, GZMA, GNLY, and PRF1 is observed, a subject may be determined to be likely to experience a systemic lupus erythematosus flare, but is not limited thereto.

In addition, a third aspect of the present disclosure provides a kit for predicting systemic lupus erythematosus (SLE) flare, comprising the composition for predicting systemic lupus erythematosus flare according to the second aspect of the present disclosure.

In addition, a fourth aspect of the present disclosure provides a method for predicting systemic lupus erythematosus (SLE) flare, comprising: isolating CD8+ T cells from a blood sample; measuring expression levels of a protein expressed in the CD8+ T cells or a gene encoding the same; comparing the expression levels of the protein or the gene with expression levels of a control sample; and predicting a systemic lupus erythematosus flare based on comparison results of the expression levels.

According to one embodiment of the present disclosure, the protein expressed in the CD8+ T cells or the gene encoding the same may comprise one or more selected from the group consisting of GZMH, GZMB, GZMA, GNLY, and PRF1, but is not limited thereto.

According to one embodiment of the present disclosure, when upregulation of an expression level of one or more of GZMH, GZMB, GZMA, GNLY, and PRF1 is observed, a subject may be determined to be likely to experience a systemic lupus erythematosus flare, but is not limited thereto.

In addition, a fifth aspect of the present disclosure provides a method for screening a therapeutic agent for systemic lupus erythematosus (SLE), comprising: treating CD8+ T cells with a candidate substance for a therapeutic agent for systemic lupus erythematosus; observing a change in an expression level of a protein expressed in the CD8+ T cells or a gene encoding the same; and selecting, as a therapeutic agent for systemic lupus erythematosus, a candidate substance that decreases the expression level of the protein expressed in the CD8+ T cells or the gene encoding the same.

According to one embodiment of the present disclosure, the protein expressed in the CD8+ T cells or the gene encoding the same may comprise one or more selected from the group consisting of GZMH, GZMB, GZMA, GNLY, and PRF1, but is not limited thereto.

The aforementioned means for solving the problem are merely exemplary and should not be construed as intending to limit the present disclosure. In addition to the exemplary embodiments described above, further embodiments may exist in the drawings and the detailed description of the invention.

### Effect of the Invention

The biomarker for predicting systemic lupus erythematosus (SLE) flare according to the present disclosure can predict flare in SLE patients more accurately than conventional biomarkers, thereby improving monitoring of disease activity in SLE patients and contributing to the development of potential new therapeutic interventions.

However, the effects that can be obtained from the present disclosure are not limited to the effects described above, and there may be other effects.

### BRIEF DESCRIPTION OF THE DRAWING

The patent or application file contains at least one drawing executed in color. Copies of this patent or patent application publication with color drawing(s) will be provided by the Office upon request and payment of the necessary fee.

FIG. 1A is a schematic overview of SLE single-cell transcriptome and T cell receptor sequence profiling. Here, CTL represents a control group, TCR represents a T cell receptor, and BCR represents a B cell receptor.

FIG. 1B is UMAP visualization of cellular composition by cell type (left) and by disease activity (right) in total cells of SLE. Here, CD4 T represents CD4+ T cells, CD8 T represents CD8+ T cells, gdT represents gamma-delta T cells, NK represents natural killer cells, cM represents classical monocytes, ncM represents non-classical monocytes, cDC represents conventional dendritic cells, pDC represents plasmacytoid dendritic cells, NF represents a non-flare state, and FL represents a flare state.

FIG. 1C shows scaled average marker gene expression of 11 cell types in total immune cells of SLE. Here, the size of each dot represents a proportion of cells expressing the marker gene in each cell type.

FIG. 1D shows proportions of immune cell types in PBMCs by disease (left) and total numbers of immune cell types (right). Here, orange stars indicate significant increases in SLE, and green indicates increases in CTL. (**: p<0.01; ***: p<0.001; ****: p<0.0001)

FIGS. 1E to 1I show scaled average expression of IFN-related genes (n=100) by disease across cell types. Here, the heatmap on the left represents the SLE dataset of Perez et al., and the heatmap on the right represents the present dataset. The adjacent bar graph shows log-scale fold changes of DEG results (Wilcoxon test) for total cells and is colored according to significance (|Log2(FC)|>log2(1.5) & p<1×10−6). The IFN-related genes are clustered into five IFN modules, and the genes on the y-axis are arranged in the order of fold change in each IFN module.

FIG. 1J shows global (top), lymphoid (middle), and myeloid (bottom) IFN module expression patterns across immune cells. Here, each IFN module shows fold change correlation between the dataset of Perez et al. and the present dataset (left), module scores for total cells (middle), and target cell types (right). (****: p<0.0001)

FIG. 2A is UMAP visualization of cellular composition of B cells by cell type (left) and by disease (right). Here, B naive represents naïve B cells, ABCs represents atypical B cells, and B memory represents memory B cells.

FIG. 2B shows scaled average marker gene expression of five subtypes in B cells. Here, the size of each dot represents a proportion of cells expressing the marker gene in each cell type.

FIG. 2C is UMAP visualization of cellular composition of CD20+B cells by cell type (upper left) and by disease (lower left). Here, the right panels show enriched expression of TBX21 as an ABC marker (top) and lack of CXCR5 (bottom).

FIG. 2D shows inferred trajectories overlaid on a UMAP plot showing two directions from B naive toward ABCs and B memory.

FIG. 2E shows density of cellular composition aligned with pseudotime.

FIG. 2F shows proportions of B cell subtypes by disease. Orange stars indicate significant increases in SLE, and green indicates increases in CTL. (*: p<0.05; ****: p<0.0001)

FIG. 2G shows a ratio of ABCs to B memory cells among total B cells by disease. (****: p<0.0001)

FIG. 2H shows scaled expression of genes related to B cell functionality. Here, the size of each dot represents a proportion of cells expressing the gene in each cell type, and the color of the dot edge indicates significance according to DEG analysis in B cells.

FIG. 2I is UMAP visualization of cellular composition of myeloid cells by cell type (left) and by disease (right). Here, cDC represents conventional dendritic cells, and ASDC represents AXL+SIGLEC6+ dendritic cells.

FIG. 2J shows scaled average marker gene expression of five subtypes in myeloid cells. Here, the size of each dot represents a proportion of cells expressing the marker gene in each cell type.

FIG. 2K shows proportions of myeloid cell subtypes by disease. Here, orange stars indicate significant increases in SLE, and green indicates increases in CTL. (*: p<0.05; **: p<0.01; ***: p<0.001)

FIG. 2L shows type I IFN production levels of myeloid cell subtypes by disease.

FIG. 2M shows correlation between an average type I IFN production level of pDCs and a type I IFN response of ABCs.

FIG. 3A is UMAP visualization of cellular composition of αβT cells by cell type (left) and by disease (right). Here, Tcm represents central memory T cells, Tem represents effector/memory T cells, Treg represents regulatory T cells, cycling represents cycling T cells, CD4 CTL represents CD4+ cytotoxic T cells, and CD8 GZMK+ represents CD8+ GZMK+ transitional T cells.

FIG. 3B shows scaled average marker gene expression of 10 subtypes in αβT cells. Here, the size of each dot represents a proportion of cells expressing the marker gene in each cell type.

FIG. 3C shows proportions of αβT cell subtypes by disease. Here, orange stars indicate significant increases in SLE, and green indicates increases in CTL. (***: p<0.001; ****: p<0.0001)

FIG. 3D shows lymphoid IFN module score levels of αβT cell subtypes by disease.

FIG. 3E is an MA plot showing genes differentially expressed in SLE CD8 T cells compared with the control group. Here, gray dotted lines indicate cutoffs of fold change (|Log2(FC)|>1.5).

FIG. 3F is a violin plot showing levels of TCR signaling (left) and T cell-mediated cytotoxicity (right) of CD 8 T cells by disease. (****: p<0.0001)

FIG. 3G shows TCR diversity of αβT cells by disease. (****: p<0.0001)

FIG. 3H is a heatmap showing differences in cell-cell interaction strength between control and SLE samples among immune cells.

FIG. 3I shows different interaction strengths from myeloid cells to CD8 T cell subtypes in MHC-I signaling (left) and Galectin signaling (right).

FIG. 3J shows ligand-receptor communication probabilities of MHC-I signaling from cM to CD8 T cell subtypes (left) and Galectin signaling from pDC to CD8 T cell subtypes (right). Empty dots indicate non-significant interactions.

FIG. 4A shows a study design of flare analysis by scRNA & scTCR-seq.

FIG. 4B is UMAP visualization of cellular composition of flare CD8 T cells by cell type (left) and by disease (upper right).

FIG. 4C shows a proportion of CD8 T cells in a flare state in each CD8 T subtype.

FIG. 4D is an MA plot showing genes differentially expressed in CD8 T cells compared with a pre-flare state. Here, a gray dotted line in the center indicates a cutoff of fold change (|Log2(FC)|>1.25).

FIG. 4E shows an inferred trajectory overlaid on a UMAP plot showing differentiation from CD8 naive to the CD8 Tem subtype.

FIG. 4F shows correlation between inferred pseudotime and cytotoxicity of CD8 T cells. Ridge plots on each side show CD8 T cell density of each group, and dotted lines of each color indicate average values.

FIG. 4G shows proportions of TCR clonotypes in CD8 T cell subtypes at each time point for each patient.

FIG. 4H is a flow diagram showing percentages of the top 10 clonotypes in CD8 T cells. Each color indicates clonotype expansion in a flare state.

FIG. 4I is a heatmap showing an overlap rate between samples. Patient numbers below the heatmap indicate sources of the samples.

FIG. 4J shows TCR clonotype frequencies in the first and second flares of patients P3 and P4. Dots are colored according to clonotype expansion in a flare state.

### DETAILED DESCRIPTION

Hereinafter, examples of the present disclosure will be described in detail so as to be easily implemented by those skilled in the art, with reference to the accompanying drawings. However, the present disclosure may be embodied in many different forms and are not limited to the examples to be described herein. In addition, parts not related with the description have been omitted in order to clearly describe the present disclosure in the drawings and throughout the present specification, like reference numerals designate like elements.

Further, throughout this specification, when a certain part is “connected” with the other part, it is meant that the certain part may be “directly connected” with the other part and “electrically connected” with the other part with another element interposed therebetween.

Throughout the present specification, it will be understood that when a certain member is located “on”, “above”, “at the top of”, “under”, “below”, and “at the bottom of” the other member, a certain member is in contact with the other member and another member may also be present between the two members.

Throughout the specification, a case where a part “includes” an element will be understood to imply the inclusion of stated elements but not the exclusion of any other elements unless explicitly described to the contrary.

The terms “about”, “substantially”, and the like to be used in the specification are used as a numerical value or a value close to the numerical value when inherent manufacturing and material tolerances are presented in the stated meaning, and used to prevent an unscrupulous infringer from unfairly using disclosed contents in which precise or absolute numerical values are mentioned to help in the understanding of the present disclosure. Throughout the present specification, the term of “step to” or “step of” does not mean “step for”.

Throughout the present specification, the term “combinations thereof” included in the expression of the Markush form means one or more mixtures or combinations selected from the group consisting of components described in the expression of the Markush form, and means to include at least one selected from the group consisting of the components.

Throughout the present specification, “A and/or B” means “A or B, or A and B”.

Hereinafter, the biomarker composition for predicting systemic lupus erythematosus (SLE) flare according to the present disclosure will be described in detail with reference to embodiments, examples, and drawings. However, the present disclosure is not limited to these embodiments, examples, and drawings.

As a technical means for achieving the aforementioned technical problems, a first aspect of the present disclosure provides a biomarker composition for predicting systemic lupus erythematosus (SLE) flare, comprising a protein expressed in CD8+ T cells or a gene encoding the same as an active ingredient.

The biomarker for predicting systemic lupus erythematosus (SLE) flare according to the present disclosure can predict flare in SLE patients more accurately than conventional biomarkers, thereby improving monitoring of disease activity in SLE patients and contributing to the development of potential new therapeutic interventions.

According to one embodiment of the present disclosure, the protein expressed in the CD8+ T cells or the gene encoding the same may comprise one or more selected from the group consisting of GZMH, GZMB, GZMA, GNLY, and PRF1, but is not limited thereto.

According to one embodiment of the present disclosure, when upregulation of an expression level of one or more of GZMH, GZMB, GZMA, GNLY, and PRF1 is observed, a subject may be determined to be likely to experience a systemic lupus erythematosus flare, but is not limited thereto.

In addition, a second aspect of the present disclosure provides a composition for predicting systemic lupus erythematosus (SLE) flare, comprising an agent capable of measuring expression levels of a protein expressed in CD8+ T cells or a gene encoding the same as an active ingredient.

With respect to the composition for predicting systemic lupus erythematosus flare according to the second aspect of the present disclosure, detailed descriptions of parts overlapping with the first aspect of the present disclosure have been omitted. However, even if such descriptions are omitted, the contents described in the first aspect of the present disclosure may be equally applied to the second aspect of the present disclosure.

According to one embodiment of the present disclosure, the protein expressed in the CD8+ T cells or the gene encoding the same may comprise one or more selected from the group consisting of GZMH, GZMB, GZMA, GNLY, and PRF1, but is not limited thereto.

According to one embodiment of the present disclosure, when upregulation of an expression level of one or more of GZMH, GZMB, GZMA, GNLY, and PRF1 is observed, a subject may be determined to be likely to experience a systemic lupus erythematosus flare, but is not limited thereto.

In addition, a third aspect of the present disclosure provides a kit for predicting systemic lupus erythematosus (SLE) flare, comprising the composition for predicting systemic lupus erythematosus flare according to the second aspect of the present disclosure.

With respect to the kit for predicting systemic lupus erythematosus flare according to the third aspect of the present disclosure, detailed descriptions of parts overlapping with the first aspect and/or the second aspect of the present disclosure have been omitted. However, even if such descriptions are omitted, the contents described in the first aspect and/or the second aspect of the present disclosure may be equally applied to the third aspect of the present disclosure.

In addition, a fourth aspect of the present disclosure provides a method for predicting systemic lupus erythematosus (SLE) flare, comprising: isolating CD8+ T cells from a blood sample; measuring expression levels of a protein expressed in the CD8+ T cells or a gene encoding the same; comparing the expression levels of the protein or the gene with expression levels of a control sample; and predicting a systemic lupus erythematosus flare based on comparison results of the expression levels.

With respect to the method for predicting systemic lupus erythematosus flare according to the fourth aspect of the present disclosure, detailed descriptions of parts overlapping with the first aspect to the third aspect of the present disclosure have been omitted. However, even if such descriptions are omitted, the contents described in the first aspect to the third aspect of the present disclosure may be equally applied to the fourth aspect of the present disclosure.

According to one embodiment of the present disclosure, the protein expressed in the CD8+ T cells or the gene encoding the same may comprise one or more selected from the group consisting of GZMH, GZMB, GZMA, GNLY, and PRF1, but is not limited thereto.

According to one embodiment of the present disclosure, when upregulation of an expression level of one or more of GZMH, GZMB, GZMA, GNLY, and PRF1 is observed, a subject may be determined to be likely to experience a systemic lupus erythematosus flare, but is not limited thereto.

In addition, a fifth aspect of the present disclosure provides a method for screening a therapeutic agent for systemic lupus erythematosus (SLE), comprising: treating CD8+ T cells with a candidate substance for a therapeutic agent for systemic lupus erythematosus; observing a change in an expression level of a protein expressed in the CD8+ T cells or a gene encoding the same; and selecting, as a therapeutic agent for systemic lupus erythematosus, a candidate substance that decreases the expression level of the protein expressed in the CD8+ T cells or the gene encoding the same.

With respect to the method for screening a therapeutic agent for systemic lupus erythematosus according to the fifth aspect of the present disclosure, detailed descriptions of parts overlapping with the first aspect to the fourth aspect of the present disclosure have been omitted. However, even if such descriptions are omitted, the contents described in the first aspect to the fourth aspect of the present disclosure may be equally applied to the fifth aspect of the present disclosure.

According to one embodiment of the present disclosure, the protein expressed in the CD8+ T cells or the gene encoding the same may comprise one or more selected from the group consisting of GZMH, GZMB, GZMA, GNLY, and PRF1, but is not limited thereto.

Hereinafter, the present invention will be explained in more detail through Examples, but the following Examples are for the purpose of illustration only and are not intended to limit the scope of the present disclosure.

### [Example 1] Profiling of the Immune Cell Landscape in SLE

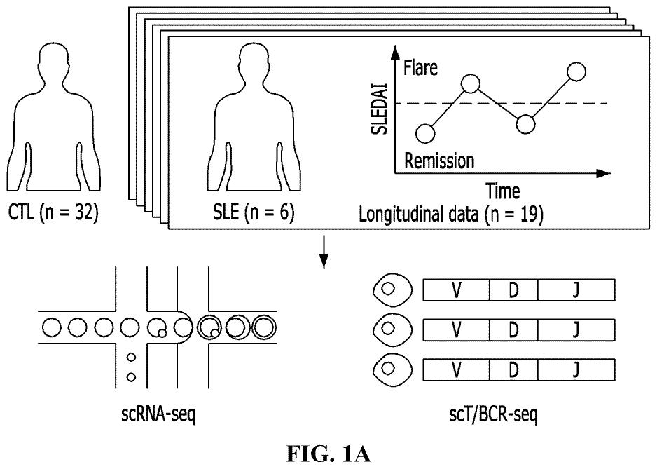

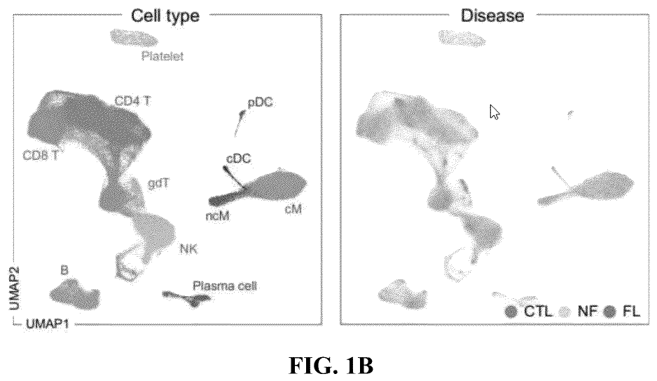

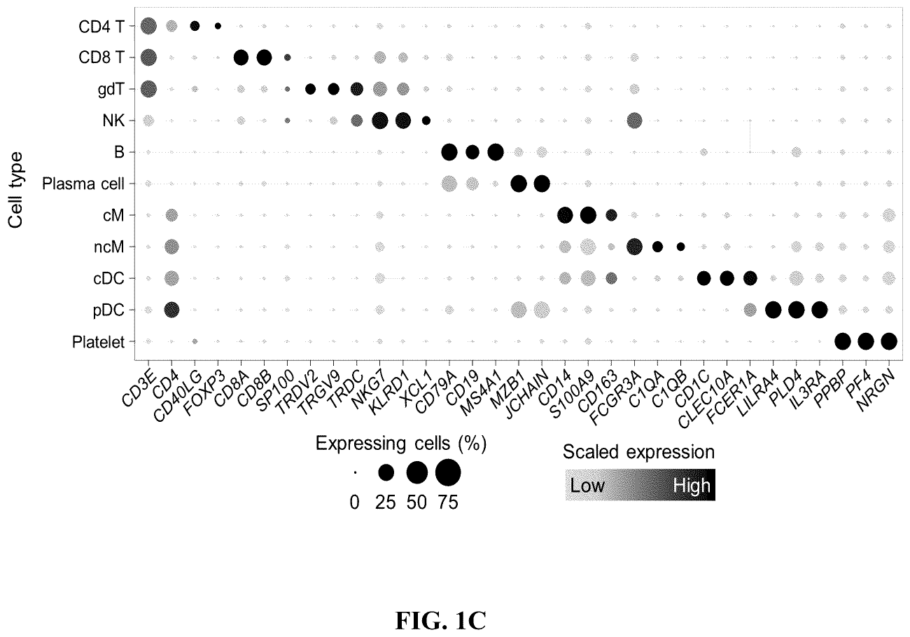

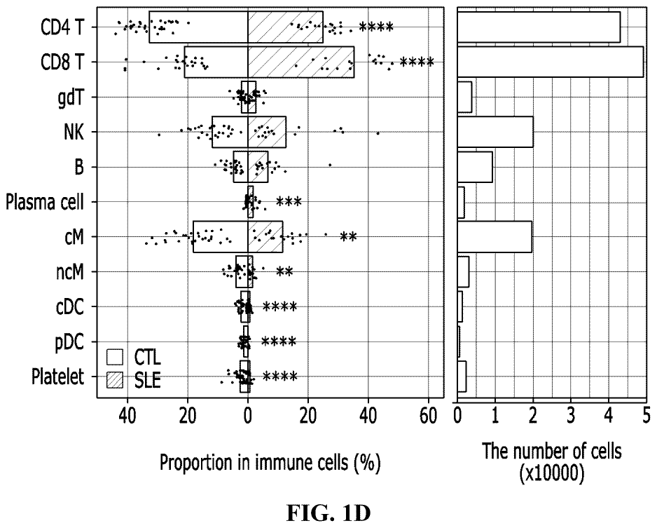

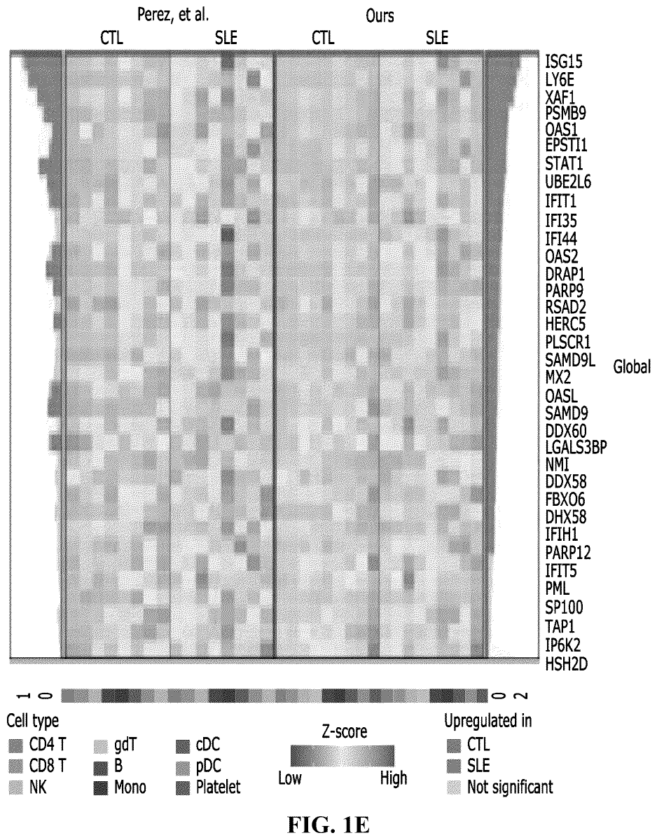

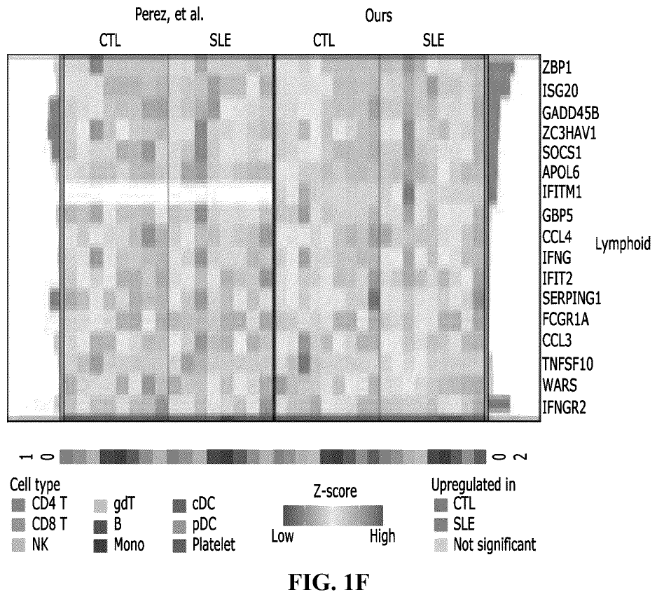

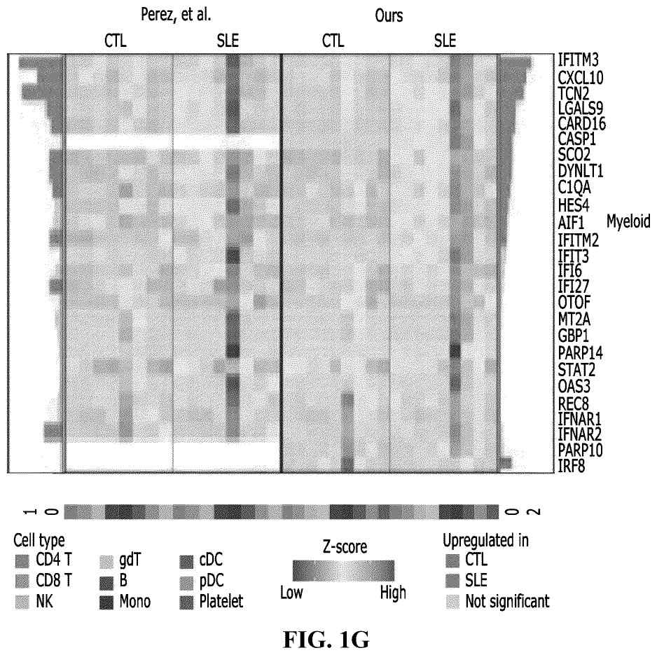

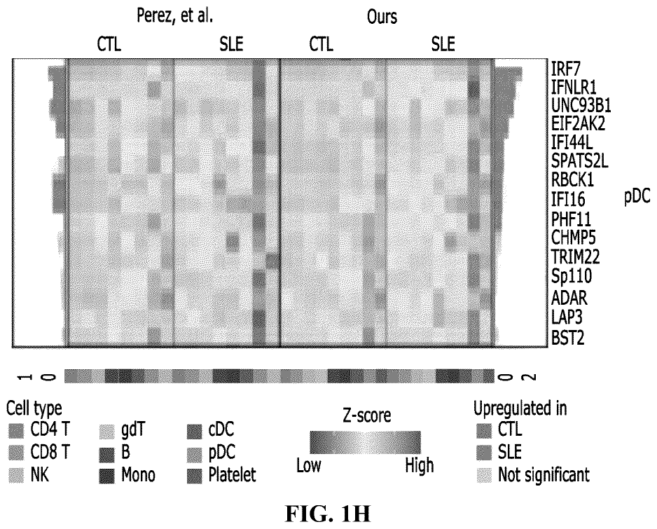

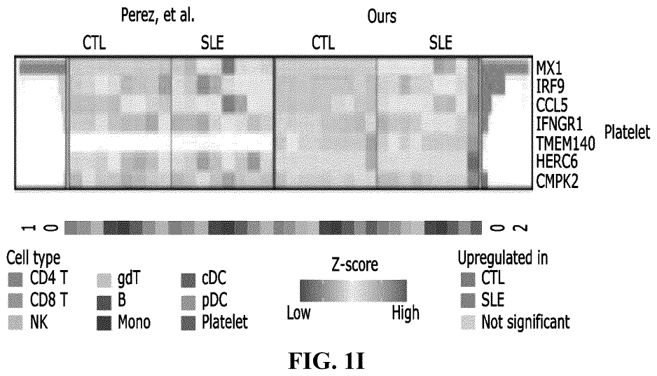

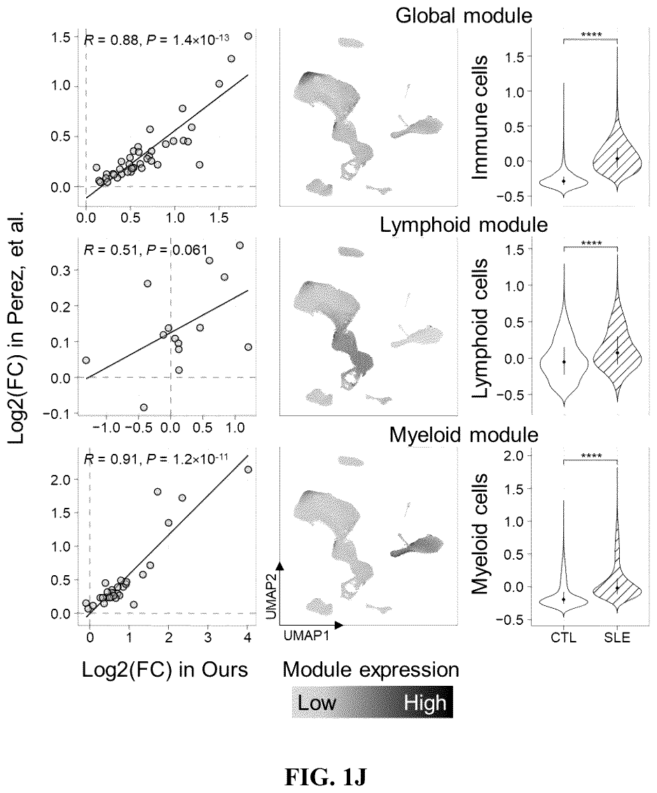

PBMCs were obtained from six SLE patients (N=19) and 33 controls at flare and remission time points during the disease course, and scRNA-seq data and scTCR-seq data were generated (FIG. 1A). The PBMCs were cryopreserved after isolation and were subsequently profiled using the 10X Genomics Chromium Droplet platform. SLEDAI scores increased from 4-14 before flare to 9-19 after flare. All patients exhibited systemic manifestations of SLE, including the presence of anti-dsDNA, decreased complement levels (C3, C4, and CH50), and renal injury characterized by proteinuria and decreased eGFR. Correlation analysis revealed significant relationships among SLEDAI scores, anti-dsDNA titers, and proteinuria levels. In particular, an increase in proteinuria during flare was associated with more severe renal injury.

A total of 164,200 cells were analyzed, excluding estimated doublets and low-quality cells. Through integration of gene expression profiles among patient samples and unsupervised clustering of cells using standard marker examination, nine major cell types were identified and visualized in UMAP space: CD4 T cells (CD3E, CD4, CD40LG, and FOXP3), CD8 T cells (CD3E, CD8A, CD8B, and S100B), natural killer (NK) cells (NKG7, KLRD1, and XCL1), γδ T cells (CD3E, TRDV2, TRGV9, and TRDC), B cells (CD79A, CD19, and MS4A1), plasma cells (MZB1 and JCHAIN), classical monocytes (cM) (CD14, S100A9, and CD163), non-classical monocytes (ncM) (FCGR3A, C1QA, and C1QB), conventional dendritic cells (cDCs; CD1C, CLEC9A, and FCER1A), plasmacytoid dendritic cells (pDCs; LILRA4, PLD4, and IL3RA), and platelets (PPBP, PF4, and NRGN) (FIGS. 1B and 1C). These annotation results showed reliable concordance with automatic annotation using Azimuth, a reference-based mapping tool.

Consistent with previous reports of PBMC scRNA-seq data, T cells were the most predominant, followed by NK cells, monocytes, and B cells (FIG. 1D). Comparative analysis of cellular composition between the control group and SLE patients revealed significant differences. In SLE samples, CD4 T cells, monocytes, cDCs, and pDCs were markedly decreased, whereas CD8 T cells and B cells were distinctly increased (FIG. 1D). Lymphopenia is a phenomenon frequently observed in SLE patients. In addition, decreases in cDCs and platelets were also observed, which have been associated with interferon responses in previous studies. These changes in cellular composition were also confirmed in an independent cohort.

To investigate this aspect in more detail, k-means clustering was performed on 100 previously identified IFN-related genes to analyze expression patterns in various cell types between the control group and SLE patients (FIGS. 1E and 1F). In differential gene expression analysis of total cells, approximately 61% of the IFN genes were overexpressed in SLE. Based on this, the optimal number of clusters was determined to be five according to silhouette scores, and each cluster was designated as an “IFN module” according to shared expression patterns within specific cell types. The Global IFN module, including ISG15, PSMB9, OAS1, STAT1, TAP1, IFIT1, SAMD9L, and the like, was upregulated in most immune cell types. The Lymphoid IFN module, including IFITM1, ISG20, SP100, SAMD9, GBP5, and the like, was mainly expressed in SLE T cells, NK cells, and B cells. In contrast, the Myeloid IFN module, including IFITM3, MT2A, IFI6, TNFSF10, SCO2, PLSCR1, MX2, and the like, was enriched in monocytes and cDCs. The pDC IFN module, including IRF7, BST2, LAP3, and the like, was particularly enriched in pDCs, and the Platelet IFN module, including TMEM140, IRF9, and the like, was mainly expressed in platelets and other immune cells. This finding was also observed in an independent cohort (FIG. 1E).

These IFN modules showed high concordance with the original groups, and the distribution of module genes identified in a previous microarray analysis showed that genes of the M1.2 module, which were independent of changes in disease status compared with M3.4 and M5.12, were mainly included in the Global and Myeloid IFN modules. This emphasizes the value of single-cell resolution analysis in revealing additional heterogeneity within populations that cannot be distinguished among cell types.

### [Example 2] Identification of B Lymphocyte and Myeloid Cell Subpopulations Exhibiting Enhanced Type I IFN responses

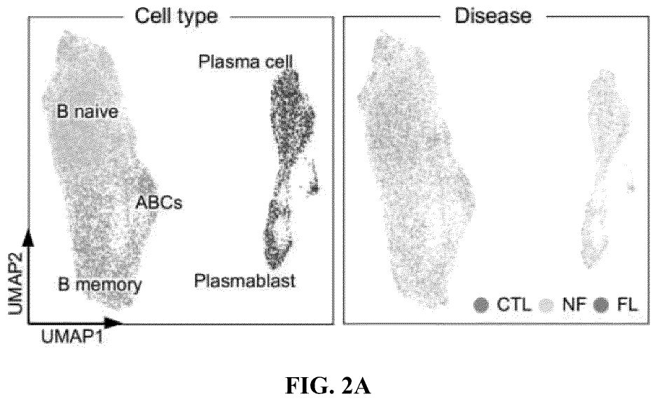

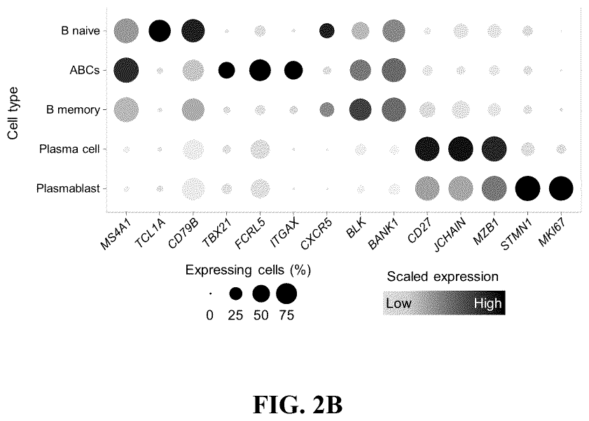

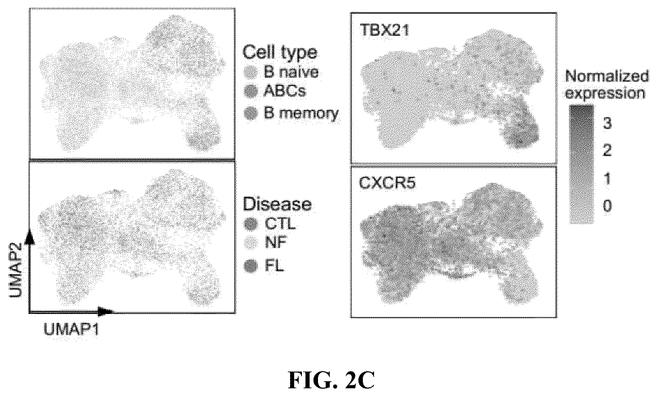

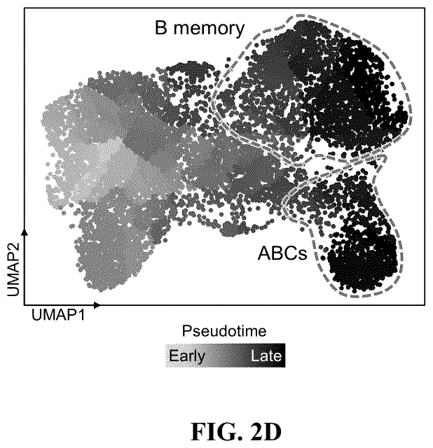

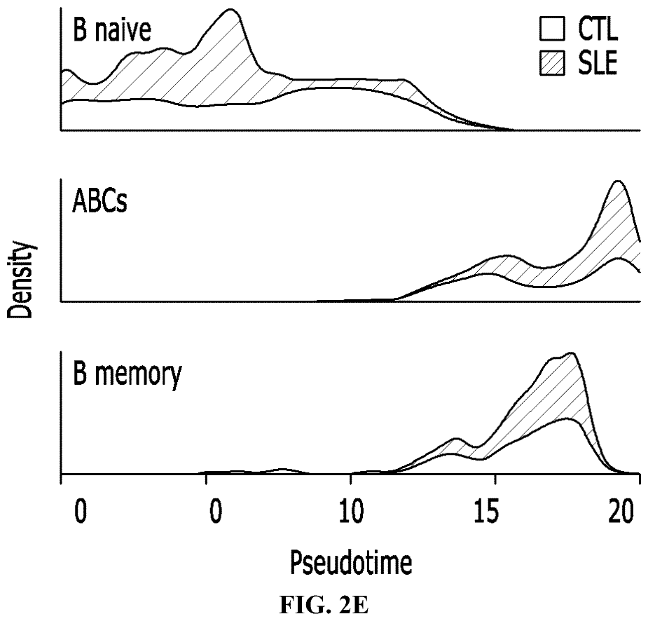

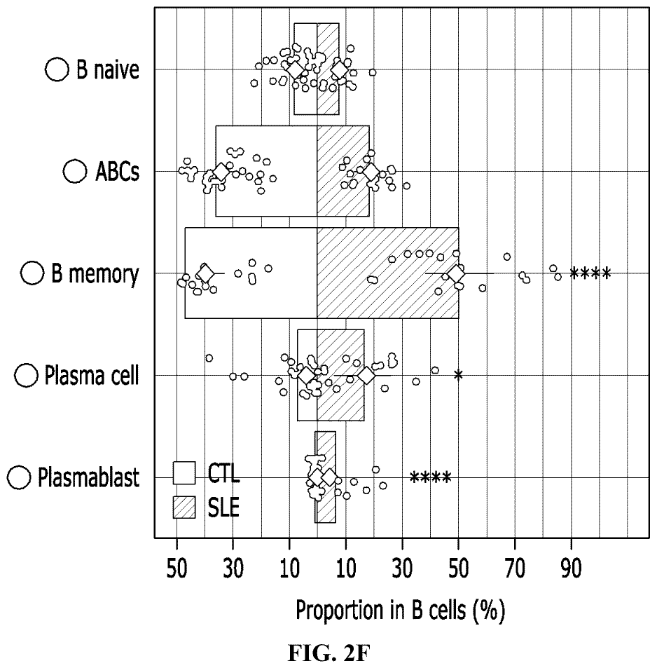

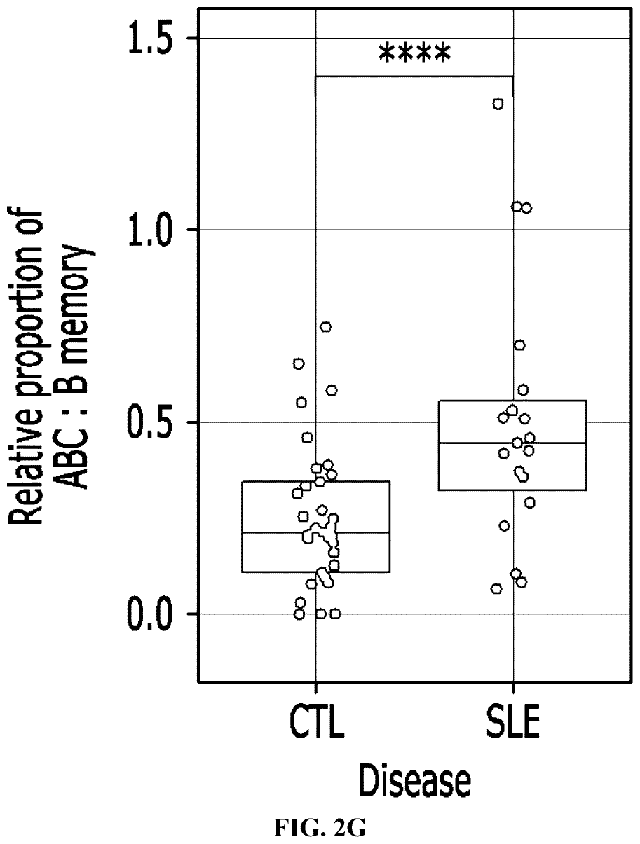

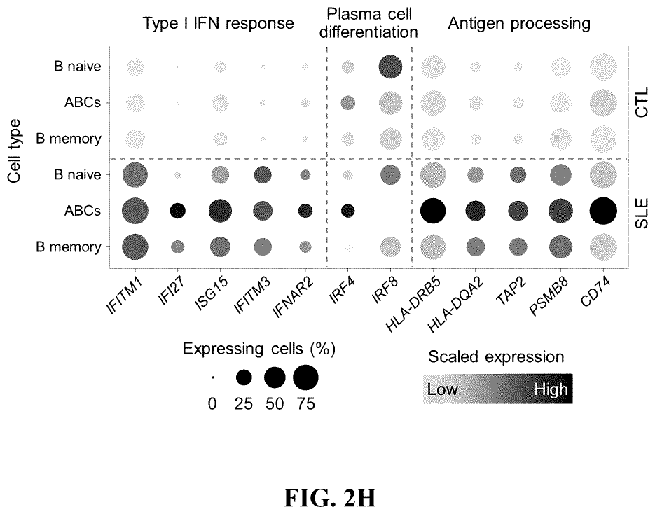

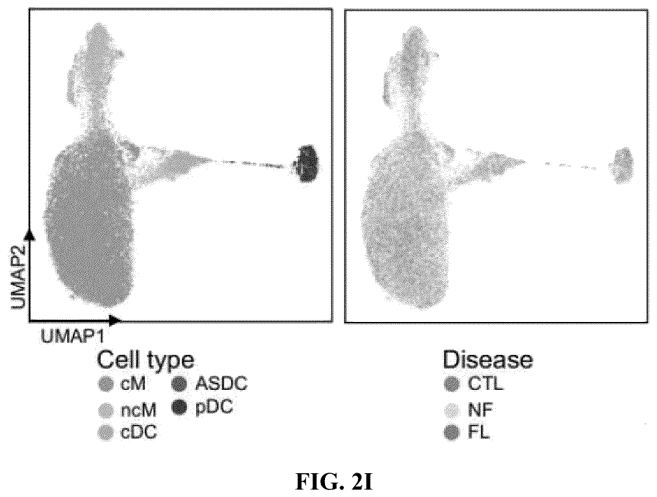

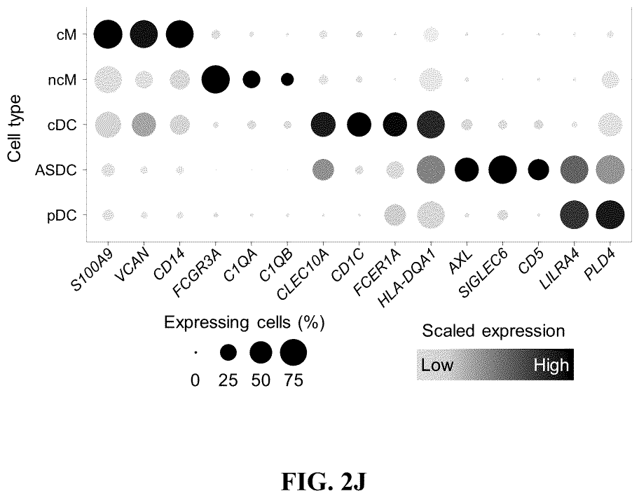

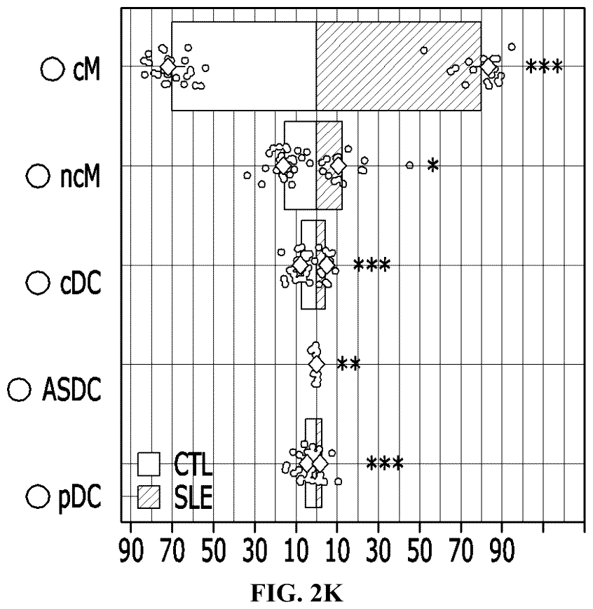

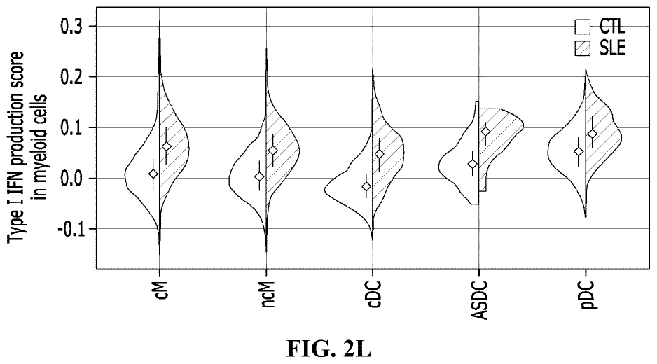

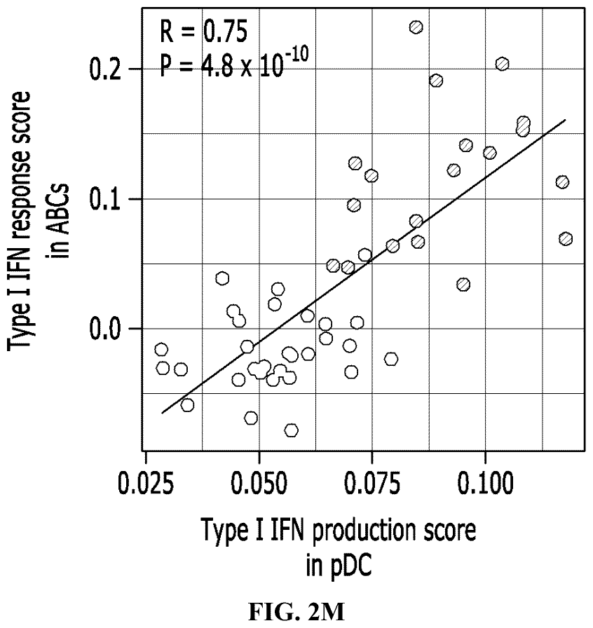

Unsupervised clustering was performed on B lymphocytes. A total of five clusters appeared within the B cell lineage, including three B cell subtype clusters and two plasma cell subtypes (FIG. 2A). These were identified by marker genes (FIG. 2B). Within these clusters, atypical B cells (ABCs), which express markers such as TBX21 (T-bet) and ITGAX (CD11c) but lack CXCR5, were identified (FIGS. 2B and 2C). Previous studies have associated ABCs with autoantibody production in conditions such as SLE and rheumatoid arthritis.

Understanding the developmental pathway of ABCs is important, particularly in view of their unique differentiation process compared with conventional B cells. To gain deeper insight into this, dynamic differentiation states and cellular transitions were explored by inferring state trajectories using Monocle3. This analysis showed that both ABCs and memory B cells originate from the naïve B cell lineage, but their trajectories diverge in different directions with similar levels of differentiation and distinct gene expression (FIGS. 2D and 2E). This observation is consistent with previous studies suggesting that ABCs mature in extrafollicular regions, whereas conventional B cells mature in germinal centers of follicular regions.

The present analysis showed marked expansion of plasma cells and plasmablasts acting as antibody-secreting cells (ASCs) in SLE (FIG. 2C). This finding is consistent with previous reports on proliferation of plasma cells in SLE. In addition, the ratio of ABCs to memory B cells was found to be significantly increased (FIGS. 2E and 2G). This suggests that SLE promotes conversion of B cells into ASCs through a non-canonical differentiation pathway. This observation is further supported by an increased expression ratio of IRF4 to IRF8 (FIG. 2H). This is particularly relevant in view of the fact that high expression of IRF4 is important for promoting plasma cell differentiation, whereas IRF8 acts to counterbalance this process. Moreover, ABCs exhibited the most prominent type I IFN response among B cell populations, suggesting that abnormal behavior of ABCs may be influenced by high IFN stimulation inherent to SLE. Using BCR sequencing data, reduced diversity of BCR clonotypes was detected in SLE, suggesting predominance of expanded clones. However, the analysis did not identify specific clonotypes associated with ABCs.

Next, it was investigated how myeloid cells are intricately involved in immune dysregulation and stimulate B cells to produce autoantibodies. Reclustering of myeloid cells identified five subpopulations: cM (cM; CD14 and S100A9), ncM (ncM; FCGR3A and TNFSF13B), cDCs (CLEC10A, CD1C, and FCER1A), AXL+ SIGLEC6+ DCs (ASDCs; AXL, SIGLEC6, and CD5), and pDCs (LILRA4 and PLD4) (FIGS. 2I and 2J). It was observed that the proportion of pDCs, which are major producers of type I IFN, was decreased in SLE (FIG. 2K). This decrease may be due to migration to inflamed tissues. Increased type I IFN production by pDCs in SLE was consistent with the IFN response of ABCs (FIGS. 2L and 2M), suggesting a potential interaction between pDCs and ABCs in SLE pathogenesis. Taken together, these results show that ABCs differentiate from the naïve B cell lineage in SLE and exhibit significant expansion and an enhanced type I IFN response, confirming their important role in ABC maturation and SLE pathogenesis.

### [Example 3] Altered Behavior of T Lymphocytes in SLE Pathogenesis

T cells are essential for systemic autoimmunity and inflammation in the pathogenesis of SLE. They not only regulate B cell responses but also infiltrate target tissues and induce tissue damage. Changes in gene expression and cytokine production caused by dysregulation of T cell signaling may consequently affect the behavior and characteristics of T cells in SLE.

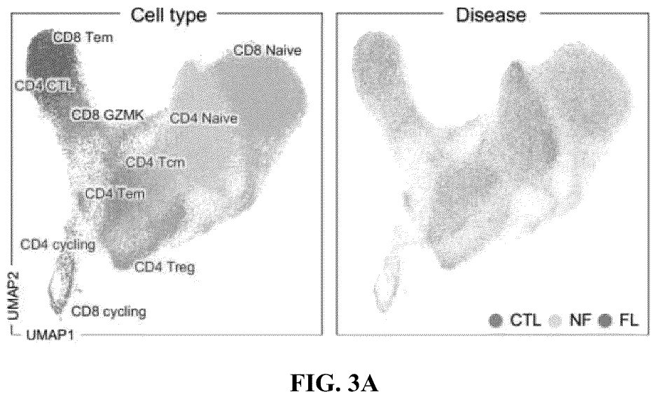

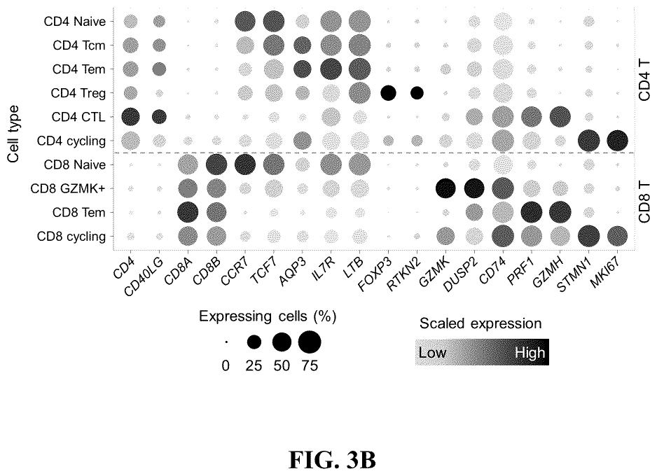

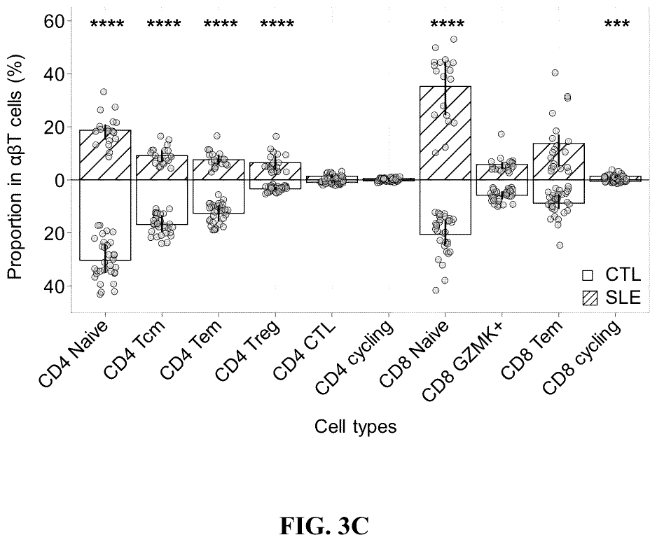

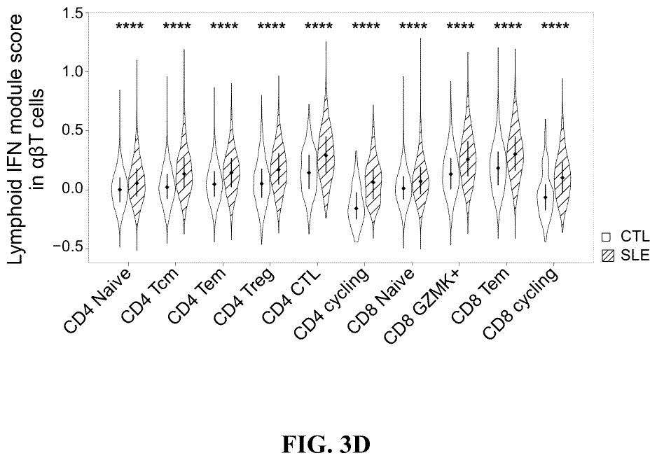

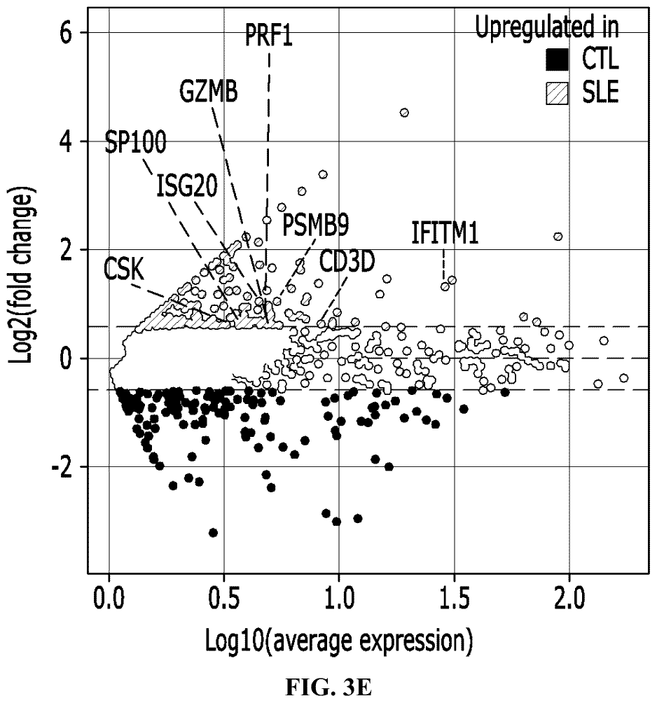

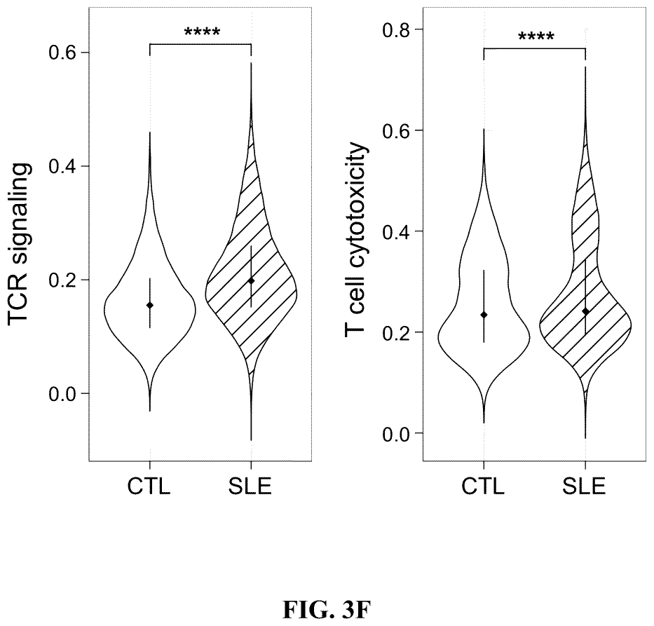

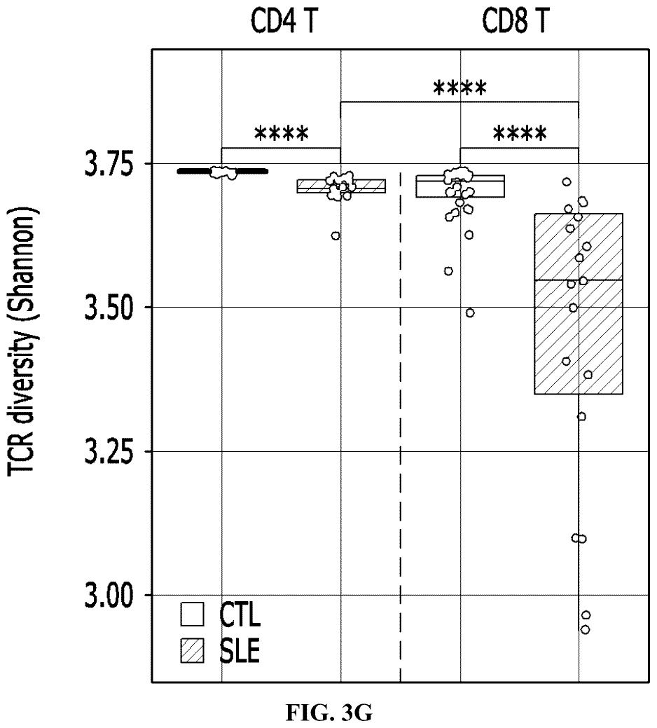

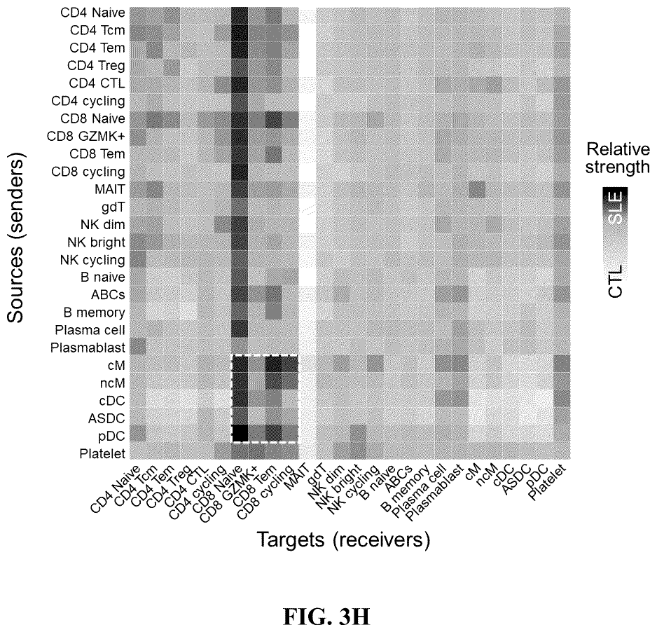

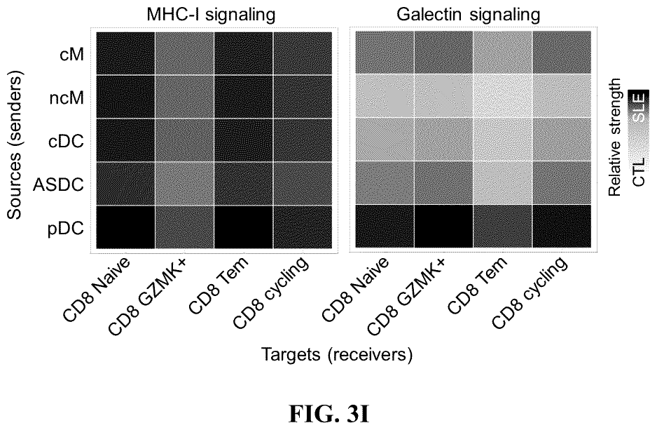

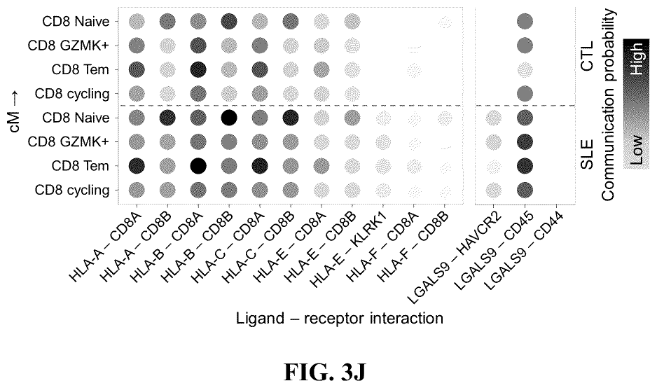

According to gene expression profiles based on established markers, six subtypes were distinguished in CD4 T cells, and four subtypes were distinguished in CD8 T cells (FIGS. 3A and 3B). Although the overall proportion of CD4 T cells was decreased in SLE, CD4 regulatory T cells (Tregs) were increased (FIG. 3C). However, CD4 Treg cells in SLE exhibited dysfunctional characteristics, particularly through downregulation of CTLA4 and IL2RA expression, which are important inhibitors of T cell activation.

This pattern was further supported by low scores of IL-2 and TGF-β signaling pathways. This suggests that the expanded CD4 Treg cell population in SLE may have a reduced ability to effectively regulate other immune cells and may fail to control autoreactive immune responses.

Unlike CD4 T cells, CD8 T cells showed an increase in SLE, particularly in naïve T cells (FIG. 3C). In addition, lymphoid IFN module scores were highest in cytotoxic T cells, such as CD8 Tem cells or cytotoxic CD4 T cells (CD4 CTLs) (FIG. 3D). Differentially expressed gene analysis showed that genes upregulated in CD8 T cells of SLE were associated with IFN genes enriched in lymphoid cells (IFITM1, SP100, and ISG20), TCR signaling (PSMB9, CD3D, and CSK), and cytotoxicity (PRF1 and GZMB). This suggests hyperactivation of cytotoxic CD8 T cells in SLE compared with the control group (FIG. 3E). In particular, increases were observed in both TCR signaling and cytotoxicity (FIG. 3F), indicating correlations between these gene set scores and disease. Consistent with enhanced TCR signaling, a significant decrease in the diversity of TCR clonotypes within CD8 T cells was observed (FIG. 3G). This indicates expansion of specific CD8 T cell clonotypes by T cell activation.

To understand molecular and cellular interactions among immune cells, a cell-cell communication network according to gene expression profiles was inferred using CellChat based on a ligand-receptor interaction knowledge base. In particular, it was confirmed that CD8 T cells participate in extensive interactions with most other immune cells (FIG. 3H). These prominent interactions were particularly evident in their main participation in MHC-I and Galectin signaling with myeloid cells (FIGS. 3I and 3J). MHC-I signaling showed the highest relative interaction strength between cM and CD8 T cells and was characterized by enhanced ligand-receptor interactions between HLA class I molecules and CD8A, CD8B, and KLRK1. This indicates enhanced activation of CD8 T cells due to stronger antigen presentation. In addition, Galectin signaling was most prominent between pDCs and CD8 T cells, mainly through interaction of LGALS9. The LGALS9 gene encoding Galectin-9 has been identified as a biomarker of SLE due to high serum protein levels in SLE patients. Moreover, the fact that this gene is included in the myeloid IFN module suggests a previously unexplored aspect of Galectin signaling in T cell activation in the context of SLE.

Taken together, cytotoxic CD8 T cells exhibited increased activation and enhanced interactions, whereas Tregs appeared to have a reduced ability to effectively regulate such activities, providing insight into the unique immune perturbations observed in SLE.

### [Example 4] Hyperactivation of Cytotoxic CD 8+ T Cells During Flare

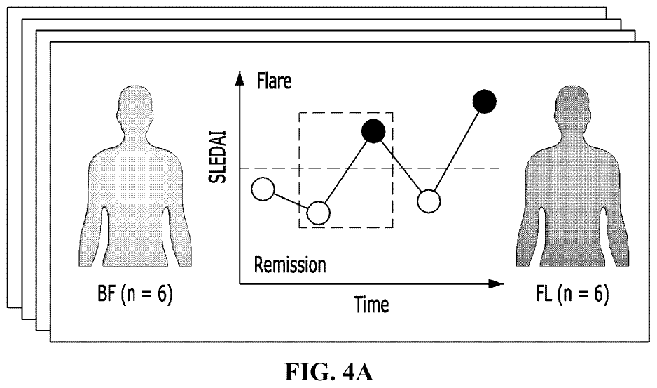

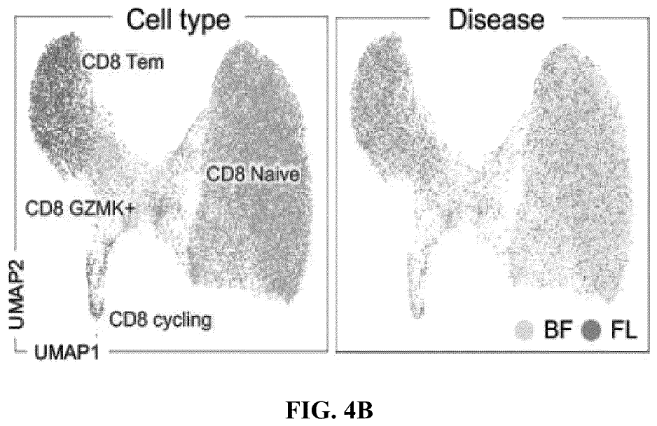

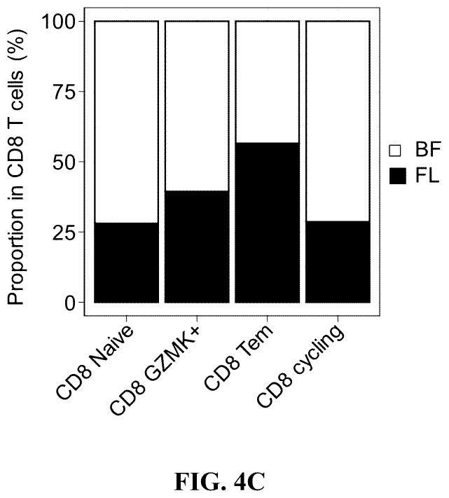

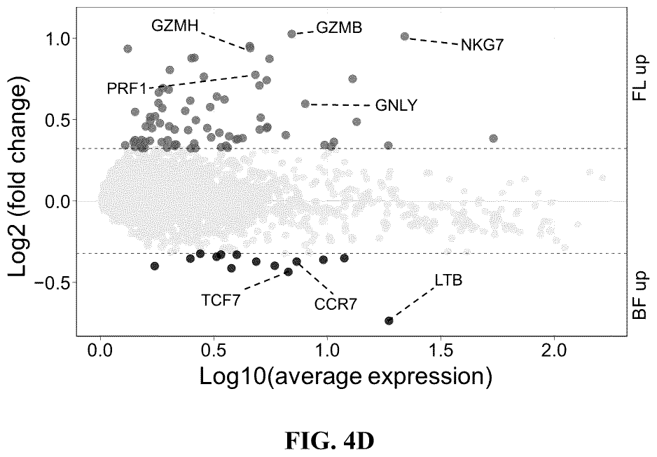

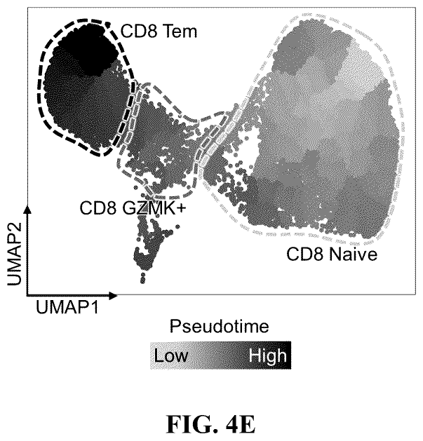

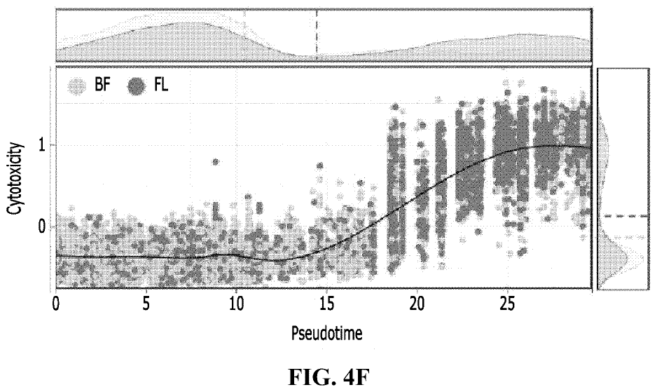

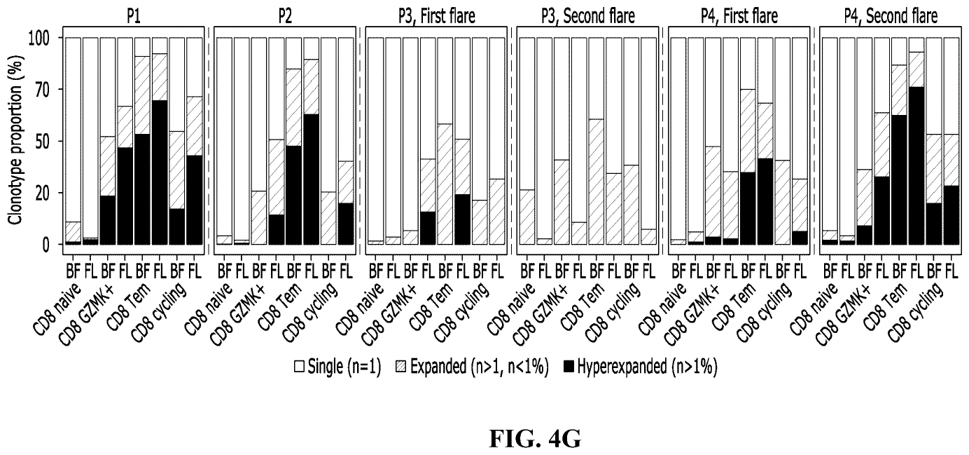

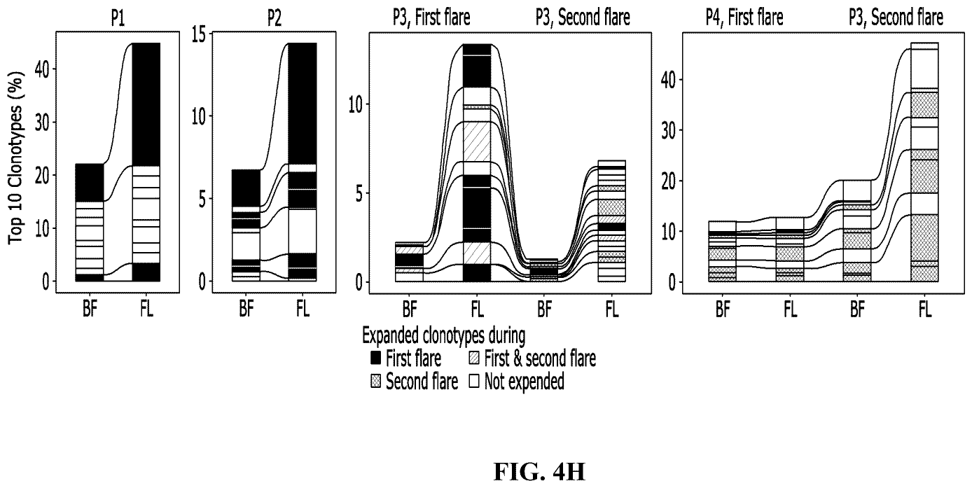

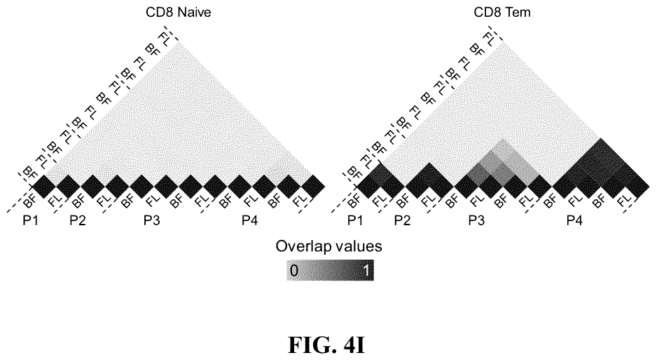

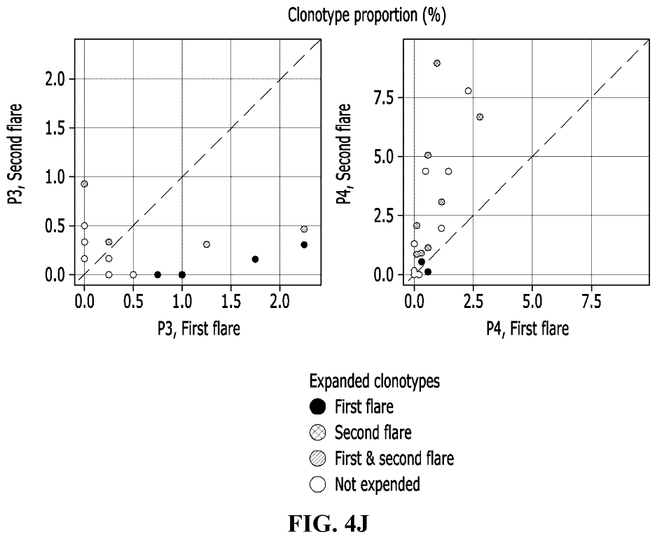

Flares in SLE represent acute exacerbations that severely impair patient well-being. Although the precise etiological basis of flares remains largely unknown, it is presumed to be an intersection of environmental, hormonal, and genetic factors. At the center of this inflammatory cascade during flare are, in particular, CD8 T cells. To analyze immune dynamics during flare, paired samples before and after flare were analyzed from four patients, two of whom exhibited two flares and remissions (total n=12) (FIG. 4A). As expected, flare showed a distinct increase in SLEDAI scores compared with the pre-flare baseline state, accompanied by an increase in proteinuria. This highlights the systemic inflammatory burden during flare.

Subclustering of CD8 T cells in paired samples before and after flare revealed three clusters, including CD8 naive, CD8 GZMK+, and CD8+ effector/memory (Tem) cells (FIG. 4B). UMAP embedding of single cells showed a distinct increase in the CD8 Tem subpopulation during the flare period (FIG. 4C). At the molecular level, flare-associated CD8 T cells showed a surge in genes related to cytotoxicity, such as GZMH, GZMB, GZMA, GNLY, and PRF1 (FIG. 4D). In contrast, representative genes of the memory phenotype, including TCF1, LEF1, and CCR7, showed downregulation. These changes in gene expression supported cellular trajectory analysis (FIG. 4E), suggesting a transition from naive T cells to Tem cells. In particular, these terminally differentiated cells became more abundant during the flare state and were accompanied by enhanced cytotoxic capacity (FIG. 4F). [Example 5] Expansion of clonally restricted cytotoxic CD 8+ T cells during SLE flare

T cells undergo clonal expansion upon antigen recognition, which is an important step in differentiation into effector cells. Such specialized proliferation ensures a robust immune response against recognized pathogens. In this context, TCR sequences of CD8 T cells were analyzed and their TCR clonotypes were mapped onto UMAP space. While focusing on potential autoantigens and exploring changes during flare, the dynamics of CD8 T cell clonotypes were investigated. By tracking CD8 T cell clones using TCR sequences, T cell expansion during the flare state was detected, with particular attention to expanded cytotoxic CD8 T cells. Cells having the same TCR genes in both α and β chains were considered to belong to the same clone. Analysis of three time points from six paired pre-flare and flare samples confirmed that the proportion of hyperexpanded clonotypes (n>1%) increased during the flare state (FIG. 4G). This expansion was particularly prominent within the CD8 Tem cell subpopulation. To track which clonotypes were dominantly expanded during the flare state, the top 10 abundant clonotypes in CD8 T cells of each patient were focused on, and some clonotypes showing a change of two-fold or more were identified (FIG. 4H). Such marked expansion of specific clonotypes highlights increased T cell activation and suggests that specific epitopes may drive such activation during flare. Further analysis of clonotype frequencies expanded to the top 30 abundant clonotypes in CD8 T cells reinforced this observation and showed a clear tendency of T cell expansion, although it was not statistically significant due to the limited number of samples. Such clonal expansion provides strong evidence of a targeted immune response to specific antigens during flare episodes.

Then, to infer similarity of autoantibodies, similarity of CD8 TCR clonotypes among patients and among flares was further investigated. As expected, TCR clonotypes varied among patients, but similarity was found within individual patients and was more pronounced in CD8 Tem cells than in CD8 naive T cells (FIG. 4I). Interestingly, analysis of two consecutive time points showed that some clonotypes were initially present but were mainly expanded during specific time points (FIG. 4J). This suggests that major antigens inducing cytotoxic T cell responses may differ among flare episodes. The dynamic nature of these antigen interactions emphasizes the need for precise therapeutic approaches capable of addressing changing antigenic stimuli that activate cytotoxic T cell responses during each flare.

The aforementioned description of the present disclosure is to be exemplified, and it will be understood by those skilled in the art that the present disclosure may be easily modified in other detailed forms without changing the technical spirit or required features of the present disclosure. Therefore, it should be appreciated that the examples described above are illustrative in all aspects and are not restricted. For example, each component described as a singular form may be implemented in a distributed manner, and components described as being distributed may also be implemented in a combined form.

The scope of the present disclosure is represented by appended claims to be described below rather than the detailed description, and it is to be interpreted that the meaning and scope of the claims and all the changes or modified forms derived from the equivalents thereof come within the scope of the present disclosure.

1. A biomarker composition for predicting systemic lupus erythematosus (SLE) flare, comprising a protein expressed in CD8+ T cells or a gene encoding the same as an active ingredient.

2. The biomarker composition of claim 1, wherein the protein expressed in the CD8+ T cells or the gene encoding the same comprises one or more selected from the group consisting of GZMH, GZMB, GZMA, GNLY, and PRF1.

3. The biomarker composition of claim 2, wherein, when upregulation of an expression level of one or more of GZMH, GZMB, GZMA, GNLY, and PRF1 is observed, a subject is determined to be likely to experience a systemic lupus erythematosus flare.

4. A composition for predicting systemic lupus erythematosus (SLE) flare, comprising an agent capable of measuring expression levels of a protein expressed in CD8+ T cells or a gene encoding the same as an active ingredient.

5. The composition of claim 4, wherein the protein expressed in the CD8+ T cells or the gene encoding the same comprises one or more selected from the group consisting of GZMH, GZMB, GZMA, GNLY, and PRF1.

6. The composition of claim 5, wherein, when upregulation of an expression level of one or more of GZMH, GZMB, GZMA, GNLY, and PRF1 is observed, a subject is determined to be likely to experience a systemic lupus erythematosus flare.

7. A method for predicting systemic lupus erythematosus (SLE) flare, comprising:
isolating CD8+ T cells from a blood sample;
measuring expression levels of a protein expressed in the CD8+ T cells or a gene encoding the same;
comparing the expression levels of the protein or the gene with expression levels of a control sample; and
predicting a systemic lupus erythematosus flare based on the comparison results of the expression levels.

8. The method of claim 7, wherein the protein expressed in the CD8+ T cells or the gene encoding the same comprises one or more selected from the group consisting of GZMH, GZMB, GZMA, GNLY, and PRF1.

9. The method of claim 8, wherein in the predicting step, a subject is determined to be likely to experience a systemic lupus erythematosus flare when upregulation of an expression level of one or more of GZMH, GZMB, GZMA, GNLY, and PRF1 is observed.
